(19) United States
(12) Patent Application Publication
(10) Pub. No.: US 2023/0296632 A1
(43) Pub. Date: Sep. 21, 2023

(54) RAPID PARATHORMONE IDENTIFICATION TEST KIT

(71) Applicant: Neurovision Medical Products, Inc., Ventura, CA (US)

(72) Inventors: Raymon Grogan, Houston, TX (US); James Lee Rea, Ventura, CA (US); Mario Navarro, Carlsbad, CA (US); Gerhart Haupt, San Marcos, CA (US)

(21) Appl. No.: 18/083,870

(22) Filed: Dec. 19, 2022

Related U.S. Application Data
(60) Provisional application No. 63/321,575, filed on Mar. 18, 2022.

(51) Int. Cl. G01N 33/78; G01N 33/543

(52) U.S. Cl. CPC G01N 33/78; G01N 33/54388; G01N 33/54346; G01N 2333/635

(57) ABSTRACT

A testing device for locating parathyroid glands comprises a module for receiving an aspirated sample of fluid and tissue taken from a suspected parathyroid gland. The module contains a first membrane containing an anti-parathyroid antibody conjugated to nanoparticles for receiving the aspirated sample, resulting in the formation of a sample/conjugate mixture. That mixture flows across adjacent membranes to a test location containing anti-parathyroid and an indicator for parathormone. Presence of a sufficient quantity of parathormone in the conjugate of aspirated sample reaching the test line is evidenced by a readable change in the test line appearance.

This application claims priority of U.S. Provisional application Ser. No. 63/321,575 filed Mar. 18, 2022.

Described and shown here is a rapid test module, a procedure for its use and a display module and reader for displaying the results obtained. The display module utilizes immunochromatographic lateral flow assay methods including test strips chemically modified to receive, react with, and provide an output signal display that is readable visually or by a specially programed reading and results display unit.

### BACKGROUND

A parathormone assay is designed to aid medical personnel in finding the location of parathyroid glands in a patient to determine if a medical procedure on the parathyroid glands is medically necessary.

The human endocrine system consists of glands which manufacture hormones that are secreted directly into the blood stream. With reference to FIG. 1, the four parathyroid glands of the endocrine system are shown located behind the thyroid gland in the center of the neck. The parathyroid glands continuously secrete the endocrine hormone parathormone (PTH) into the blood. PTH is only found at high concentrations in the parathyroid gland with concentrations greater than 250 pg/ml compared to approximately 65-85 pg/ml in whole blood, and <30 pg/ml in other tissues. PTH circulates quickly throughout the body and is broken down within minutes in the peripheral tissues. Parathormone regulates the movement of calcium out of the bones. A serious morbid condition is caused by excess secretion of parathormone by an enlarged parathyroid gland triggered by an adenoma, a small benign tumor on one or more of the four parathyroid glands. These tumors can lead to a condition referred to as hyperparathyroidism. This condition can cause bone fractures or kidney stones and is clinically presented as an elevated blood calcium level with symptom of hypercalcemia. When medical treatment fails surgical removal of the adenoma is required.

An initial problem in performing parathyroid surgery is locating the parathyroid glands. The operations can be simple once the adenoma is located but may be complicated by the need to locate and identify all four parathyroid glands. These glands can be very small as well as anatomically variable as well as located almost anywhere in the neck or chest. The medical device industry has attempted to assist surgeons with various parathyroid identification and location technologies, including, nuclear medicine scans, near infra-red laser locating devices, such as PTEye™ device provided by Medtronic, near infra-red cameras, intravascular markers (such as methylene blue and others), serum parathormone immunoassays, ultrasound visualization, and biopsy and histopathology.

Perrier and Grogan (Perrier; N D et al, “Intraoperative Parathyroid Aspiration and Parathyroid Hormone Assay as an Alternative to Frozen Section for Tissue Identification, World J. Surg. 2000; 24, pp 1319-22) reported a technique for parathyroid identification based on large difference in PTH concentration between parathyroid glands and normal tissue. They performed needle aspiration sampling of tissue suspected to be parathyroid gland and quantitative measurement of the hormone in the aspirate using serum PTH immunoassay (IOPTH) systems available in the hospital lab.

### BRIEF DESCRIPTION OF FIGURES

FIG. 1 is a drawing of a portion of the human anatomy showing the normal location of the four parathyroid glands.

FIG. 2 is top schematic perspective view of a test module for parathyroid location incorporating features of the invention described herein.

FIG. 3 is a top schematic view of the test module of FIG. 2.

FIG. 4 is a side view of the test module of FIG. 2 cut along line 4-4 of FIG. 3 with the positioning of the membranes being spaced apart for clarity.

FIG. 5 is a schematic side expanded view of the components located within the test module as shown in FIGS. 2-4.

FIG. 6 is a drawing showing a side view of the overlapping membranes on a backing card along with the lengths of the components within the test module.

FIG. 7 is a drawing showing a top view of the overlapping membranes as shown in FIG. 6.

FIGS. 8A, 8B and 8C illustrate the three stages of the flow assay which occur within the module of FIG. 2.

FIG. 9 is a photograph showing a top view of the test module of FIG. 2.

FIG. 10 is image showing a top perspective of a reader used for viewing the test results obtained using the test module described and illustrated herein.

FIG. 11 is a top view of the reader of FIG. 10 with the test module described herein inserted in the reader of FIG. 10.

### DETAILED DESCRIPTION

The rapid parathormone identification and test kit described herein uses an immunochromatographic lateral flow assay cassette which provides a rapid means to locate the parathyroid glands while in the surgical suite by detecting differences in extracellular concentration of parathyroid hormone and confirm the location of parathyroid glands. Needle aspiration samples from tissue requiring identification as parathyroid gland or not and the quantitative or semiquantitative measurement of the hormone in serum PTH is performed using a lateral flow assay cassette which allows visual reading and immediate access to the results.

With reference to the Figures, the lateral flow test described herein incorporates anti-parathyroid (antibody A) conjugated to particles (gold nanoparticles) 62 deposited on a conjugate pad. A liquid aspiration sample of fluid containing tissue 60 believed to be from a parathyroid gland (i.e., containing high PTH concentration), which is usually about one μL in volume, is injected into the sample port 23 onto a sample pad membrane (the sample pad 30) in the test module 20. Approximately 0.14 ml of a sterile fluid, such as Lactated Ringer's Solution, or a suitable alternative sterile diluent liquid, is drawn into a syringe, which can be the same sampling syringe, and injected into the sample port 23 and onto sample pad 30. This creates capillary flow along the nitrocellulose or equivalent membrane. The sample diluted by the Lactated Ringer's Solution then flows into the conjugate pad which includes a dispensed gold-antibody A conjugate 62, where the sample with resuspended conjugate particles and PTH in the sample will bind with the anti-PTH on the conjugate. This sample/conjugate mixture, which is loosely applied to the pad, then flows in to a third membrane (the nitrocellulose membrane 28). The flowing aspirate sample 60 passes over a test line 25 comprising anti-parathyroid (antibody B) 64 which is tightly adhered to the nitrocellulose membrane 28, and which results in formation of a “chromatographic sandwich” of anti A-conjugate-PTH-anti B which, with a sufficient amount of PTH, becomes visually detectable or digitally interpreted using a reader 40 described herein below. The detectable appearance of the test line 25 alerts the surgeon that the aspirate sample 60 was removed from a parathyroid gland. A generic antibody (Antibody A) 66 directed to the antibodies of the host animal is also applied as the control line 26 on the nitrocellulose membrane 28 to verify that the test has been executed correctly.

The test module 20 is, in a preferred embodiment, a two-part plastic cassette with several specially treated, wicking membranes sandwiched in an overlapping manner between an upper cover 21 and a lower cover 32. The plastic cassette is preferred because the test module is a one-time use, disposable product, the plastic is inexpensive and can be readily discarded. The upper cover has an opening therein, a sample port or well 23, for receiving fluid aspirated (analyte 60) as described below from the target tissue (a suspected parathyroid gland). The fluid analyte 60 is deposited through the sample port 23 onto a first internal membrane strip (sample pad 30). An elongated observation port 24 is positioned further along the module 20 over a third membrane strip 28, said third strip 28 displaying a colored image evidencing the presence in the analyte 60 sample of parathormone. If the colored image is detectable it is an indication that the analyte sample 60 was withdrawn from a parathyroid gland.

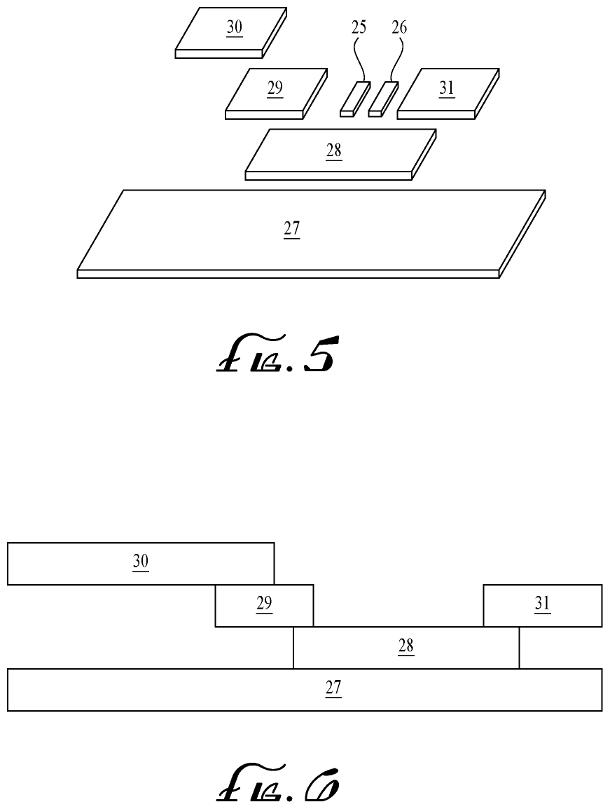

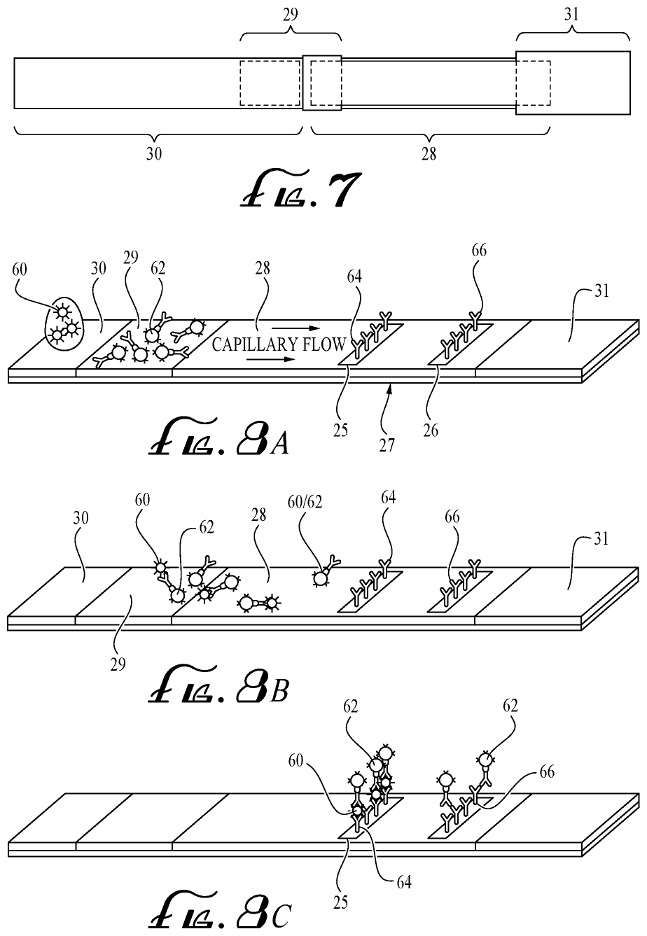

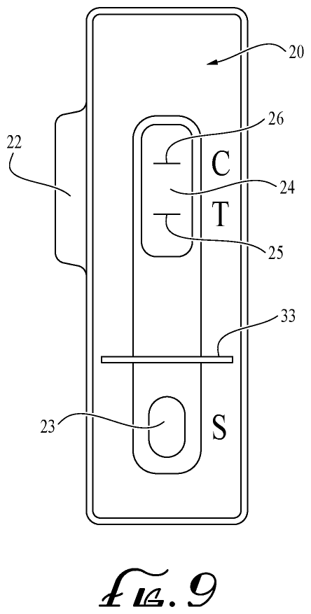

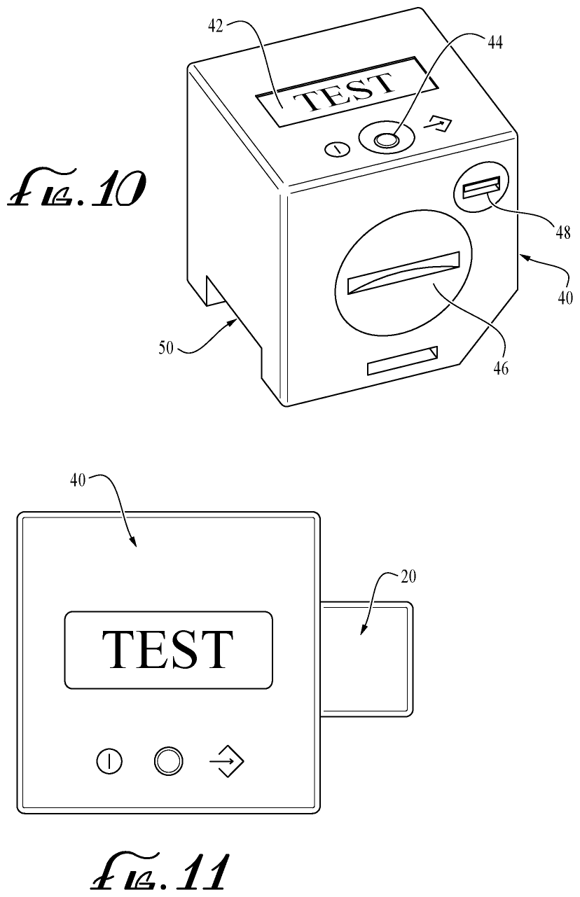

With reference to FIGS. 4-8, the multiple overlapping strips comprise wicking (by capillary action) membranes which function to transport the analyte 60 sample from a first end of the sample pad 30 through a second strip (the conjugate pad 29) and a third membrane strip (the nitrocellulose flow strip 28) to a fourth membrane (the wicking pad 31). As shown in FIGS. 5-8 each of the overlapping pads have a first end to receive the analyte 60 sample as it flows along the membrane strips and a second end to transport that analyte 60 to the next adjacent, overlapping strip or pad. The central portion of the third strip (the nitrocellulose flow strip 28) displays the result of the analysis on the test line 25 and control line 26. The several membranes or pads are supported on a backing card 27 which rests on an internal surface of the cassette bottom 32.

In a preferred embodiment the sample pad 30, which comprises a Millipore® fiber-glass membrane about 30 mm long, receives the analyte 60 sample at a first end which flows to the second end of the sample pad 30. About 15-20% of the second end of the sample pad 30 overlaps a first portion of the second pad (the conjugate pad 29 described below), also a fiber-glass membrane. A second portion or end of the conjugate pad 29 overlaps a first end of the third strip of a wicking membrane material, preferably a nitrocellulose membrane 28 (obtained from MDI) which is about 25 mm long. Positioned spaced apart on top of the 3^{rd} membrane 28 and visible through the viewing port 24 are a test line 25 and a control line 26. Further along the 3rd membrane and on top of a portion of the second end the nitrocellulose membrane 28 is the wick pad 31, which is a cotton chromatography paper (obtained from Ahlstrom), which functions to draw the analyte 60 through the module 20. The central portion of the nitrocellulose membrane 28 (the portion between the conjugate pad 29 and the wick pad 31) is visible through the viewing port 24 on the module 20.

The preferred materials and chemical treatment of the several wicking membranes shown in the figures are described below. While the specific materials described below are preferred materials one skilled in the art will recognize that many other suitable materials will have similar wicking properties and can be used in place of the preferred materials.

The sample pad 30 comprises a Millipore® fiber glass membrane selected for its wicking (by capillary action) capability. A syringe filled with an aspirated sample of the analyte 60 in 140 μL of lactated Ringer's solution or other matrix which provides for dispersion, collected as described below, is deposited through the sample well 23 in the cassette, the analyte 60 sample wicking, without changing the concentration of the analyte 60 along the length of the sample pad 30 and into contact with the second Millipore® fiber glass membrane (the conjugate pad 29). Both the sample pad 30 and the conjugate pad 29 receive a pretreatment consisting of 5 mM borate, 1% Tween® 20, 0.25% PVP-40, 0.5% BSA, in pH 8.5 water.

The second pad (the conjugate pad 29), also a Millipore® fiber glass wettable membrane pretreated as set forth above is allowed to dry and is then sprayed using an air jet with the conjugate solution which comprises anti-PTH antibody conjugated to gold nano-shells suspended in a conjugate diluent comprising 0.5% PBS, 0.5% casein, 0.5% BSA, 1% Tween 20, 0.05% azide in water and again dried. The conjugate composition reacts with the parathormone in the analyte 60, as it wicks from the sample pad 30 and onto the conjugate pad 29 to provide a reactant which provides an observable display. The antibodies conjugated to the gold nano-shells 62 bind to the PTH in the analyte 60 sample. Buffer components of the diluent and pretreatment (PBS and borate) provide stability of the antibody and normalize the pH of the sample. The buffer detergents (Tween 20, PVP) help with wetting of the conjugate pad 29 and release of the reacted conjugate, the proteins (BSA, casein) provide stability and reduction of non-specific binding of the antibody; the azide is an anti-microbial.

The reactant then flows onto the third membrane, the nitrocellulose flow strip 28, which sits blow the viewing port 24 in the module 20. A color change or intensity of the reactant display is observable by the naked eye, or preferably by a reader 40 described below, evidencing a concentration of the parathormone reactant sufficient to positively indicate that the tissue sampled is in fact a parathyroid gland. A positive result is evidenced by a readable display in the viewing port 24 on or adjacent the test line 25. A change in the appearance of the control line 26 indicates a suitable sample was collected and run through the test device. The observable display adjacent the test line 25 indicates that a parathyroid gland was located. On the other hand, the absence of that display with an observed change to the control line 26 indicates the test protocol was completed but the tissue sampled was not a parathyroid gland. The design of the lateral flow device is such that parathyroid hormone levels below 30 pg/ml do not result in a visual indication at the Test Line. However, concentrations above 30 pg/ml provide result in a darkened line that becomes progressively darker up to 250 pg/ml, a concentration that is recognized as s a positive indication that the aspirated tissue is from a parathyroid gland.

On top of a portion of the second end the nitrocellulose flow strip 28 is the wick pad 31 comprising a fourth wicking membrane, composed of a cotton chromatography paper, which functions to draw the analyte 60 through the module.

The reader 40 can be used to display the results. The reader 40 transmits a red light while the test and control lines 25, 26 are green-gray. The gold nano-shells attached thereto produce a green-gray color when immobilized on the nitrocellulose membrane 28. The test line 25 includes antibodies conjugated to the gold nano-shells 62 which binds to PTH in the analyte 60 sample. A “sandwich” is produced with the PTH at the test line 25 and the gold nanoparticle immobilized and visible due to the conjugation with the attached antibody. In contrast, the control line is formed by attaching anti-goat antibodies 66 (the antibody conjugated to the nanoparticle is sourced from goats). Any unbound and unreacted conjugated antibodies from the conjugate pad 29 flowing over the control line 26 are bound and immobilized at the test line 25 and are visualized as a differently appearing indicator.

Alternate nanoparticles that could be conjugated to the conjugate antibody for placement on the conjugate pad 29 include various different gold nano-shell sizes, europium particles, latex beads, etc. which would perform the same function but would provide a different display with a color change being based on the particle material, particle size or dyes. The particular embodiment of the assay display was developed using nanoComposix's 150 nm gold nano-shells, which provides a specific color due to particle size. However, based on the teachings herein it would be obvious to one skilled in the art that an equivalent but different change in color can be obtained by selecting nanoparticles which incorporate different chemistry, have different optical properties, and therefore provide a different performance profile. Further, it is possible for the nanoparticles to reflect light of a frequency in a non-visible range detectable by IR or UV detectors.

With reference to FIGS. 10-11, the reader 40 is a mobile measuring device provided by Chembio Diagnostics Systems specifically configured to receive the cassette 20 described herein and programed for the rapid qualitative, semi-quantitative or quantitative evaluation of diagnostic tests of tissue samples using a lateral flow assay to identify whether tissue aspirated (the analyte 60) was from the parathyroid gland. The software program in the reader 40 can be configured with specific parameters concerning the geometry of the cassette 20 and the test and control lines 25, 26 on the flow strip 28 as well as specific settings to generate an output evidencing the concentration of the target analyte 60. More specifically the reader 40 is programed with a dose-response curve for quantification and runs an algorithm wherein a reflection intensity reading is compared to the curve to provide a display 42 indicating the quantified PTH concentration (a positive PTH indication).

The green-gray color of the test and control lines 25, 26 is due to the immobilization of the gold nano-shells. The reader 40 that is placed over the viewing port 24 shines the red light through the cassette viewing port 24 and onto the third strip (the nitrocellulose flow strip 28). While the particles on the flow strip 28 reflect green light, the red light is absorbed by the particles on the flow strip 28 or is reflected by the white nitrocellulose membrane 28. A photosensor in the detector then senses the intensity of reflected light, on a scale of 0 to 250 with a 0-reflectance evidencing complete reflection and a negative reading and 250 indicating complete absorption and a positive PTH result.

The amount of PTH in an analyte 60 sample correlates positively to the number of nano-shell conjugates which are bound to the conjugated antibody 62 and which absorb or reflect the incident light (correlating with the intensity of the reflected light). The intensity of reflection from the test line 25 is due to the number of particles that become immobilized thereon and thus directly correlate with concentration of the parathormone in the analyte 60, allowing calculation of the amount of PTH in an analyte 60 sample to positively identify the tissue sample using intensity values. The reader 40 contains a chip which is programed to receive the reflectance data from the module 20. The reader 40 includes an algorithm and a dose/response curve to provide a qualitative/semi-quantitative result (e.g., “parathyroid tissue” or “<250 pg/mL”) and then display a numerical value and/or a display or signal indicating whether the tissue sample was from the parathyroid gland. While a user can visually observe the color intensity changes and reach a subjective conclusion of tissue identity, the reader 40 provides a definitive display as to whether the tissue sampled was taken from a parathyroid gland.

The Parathormone Testing (PT-ID) Procedure is as follows:
1. 140 μL lactated Ringer's solution (LRS) is drawn into an “insulin” syringe, or an equivalent sized syringe having precise volume gradations having an attached 25 or 26 Ga needle.
2. Tissue from a suspected parathyroid gland is then aspirated into the syringe containing LRS by performing multiple passes of the syringe through the tissue while applying negative pressure by withdrawing the plunger of the syringe.
3. A PT-ID cassette 20 as described herein is place face-up on a flat surface. In an alternative method, the 25 or 26 Ga needle is used to sample the tissue without attached syringe and negative pressure aspiration. This “dry tap” needle is then attached to a syringe and 0.14 ml of fluid is drawn into the syringe to wash the needle.
4. The electronic reader 40 (described herein) is placed over the viewing port 24 of the cassette 20. Alternatively, the display in the viewing port 24 can be visually observed.
5. The entire volume of the syringe (the analyte 60) is feed into the cassette 20 sample well 23 while simultaneously activating the electronic reader 40 by pressing the on-off button 44.
6. The analyte 60 sample is allowed to incubate while the electronic reader 40 measures the results. A positive result will appear at the test line 25 and on the reader in 3-5 minutes while a negative result is displayed at the control line 26 in about 5 minutes. A change to only the Control Line (C) 26 indicates to the user that the lateral flow has occurred properly but no PTH was detected. A change to the Test Line (T) 25 in addition to the Control Line (C) 26 indicates that PTH in excess of 250 pg/ml was present in the analyte 60. This presumptively identifies the aspirated tissue as being collected from a parathyroid gland. If the reader is used, the PT-ID cassette 20 is removed and the test line read visually. Alternatively, if the reader is not used the test line is read visually. In either event, readings are done precisely at 5 minutes after added the aspirate to the cassette.
7. The PT-ID cassette 20 is then discarded and the electronic reader 40 is turned off, resetting the reader to a null reading.

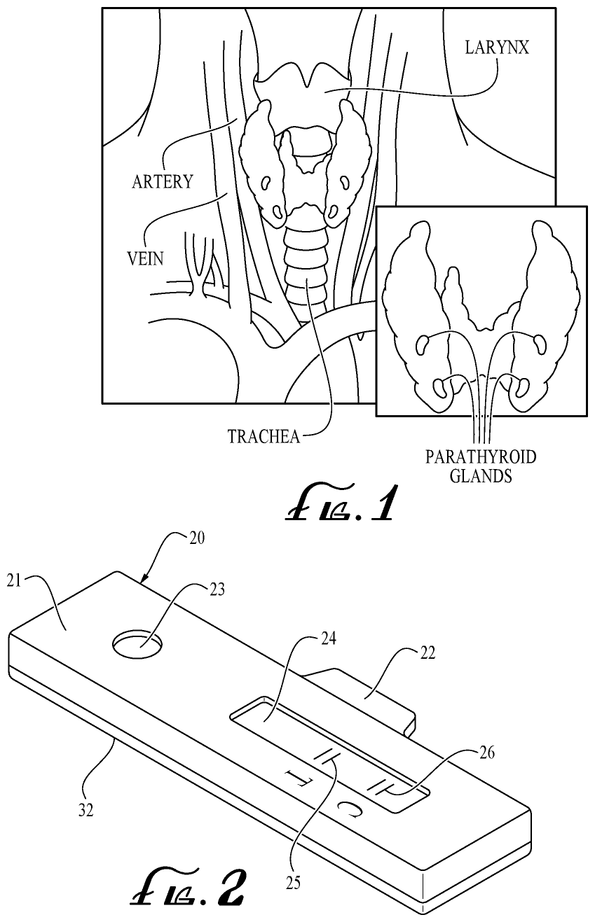

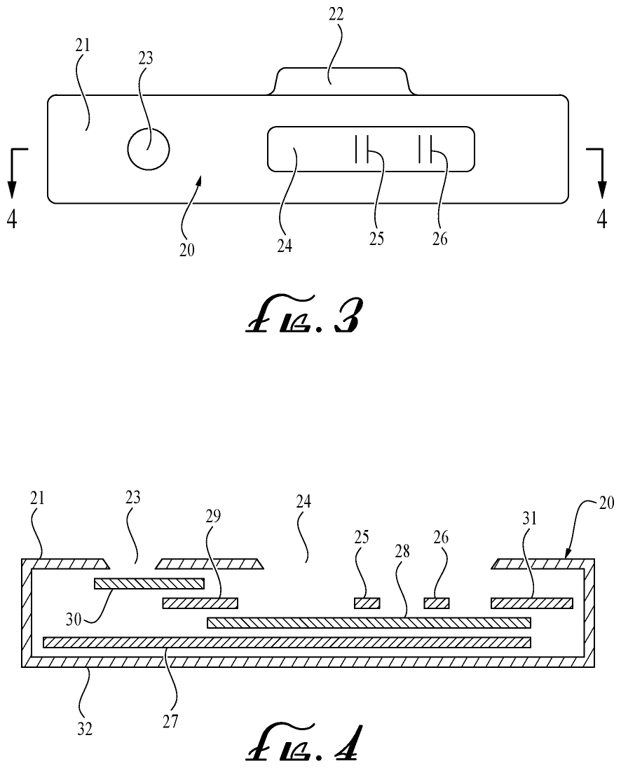

As shown in FIG. 1, a normal parathyroid gland is very small gland 8 mm or less that in length attached to the back of the thyroid. These glands are at risk for injury during thyroid surgery. In addition, they often have to be removed due to parathyroid disease. Because of their small size, color, and location, the parathyroid glands are often indistinguishable from thyroid nodules, fat, and lymph nodes, making them difficult to identify. That is why intraoperative biopsies are frequently used and frozen sections are prepared as adjuncts to confirm that the glands have been correctly identified during the surgical procedure. However, a negative aspect of preparing frozen sections is that the procedure can take at least 20 minutes to obtain the results, thus significantly delaying the surgical procedure, particularly if the biopsy turns out to have not located the parathyroid gland. The presently described test module thus allows the process to be speeded up significantly and is thus an invaluable addition to performing parathyroid surgery in the operating room.

The PT-Id module 20 is essentially an adjunct to clinical suspicion that performs a similar function to frozen section, only with a much faster turnaround time. The adoption of the testing module and assay procedure described herein, if routinely used as part of parathyroid surgery, makes these operations faster and safer. The fine needle aspiration performed by a surgeon under direct visualization provides an extremely safe assay with no risk to the patient. For comparison, fine needle aspiration under ultrasound guidance is performed routinely on the neck hundreds of thousands of times a year in the United States with virtually no complications. The information gained from the assay can help guide intraoperative decisions and provides a rapid confirmation to support the surgeon's clinical knowledge and suspicion of parathyroid gland location.

The procedure is extremely accurate and very helpful in rapidly confirming the location of parathyroid tissue. Comparative studies have shown that prior techniques to locate a parathyroid gland are highly inaccurate. However, under the same conditions the presently described testing module 20 and sampling techniques are 100% accurate.

Takeshi et al. (Takashi et al., “Near Infrared Florescence Imaging in the Identification of the Parathyroid Gland in Thyroidectomy, Laryngoscope; 131(5) (May 2021), pp 1188-1193) reports on a study of 36 patients undergoing thyroidectomy. They were subjected to surgeon identification of parathyroid gland by histopathology examination of the tissue after biopsy. The surgeon's ultimate success was 61.0% accuracy with visual identification alone and 82.9% accuracy with the NIFI (Near Infrared Fluorescence Imaging) device being used to assist the surgeon.

Evaluation of the use of the combination of the cassette module 20 and reader 40 described herein by aspirating twenty-five analyte 60 samples from patients, which were verified by histopathology, correctly identified 16 of the samples as being taken from the parathyroid, the other 9 samples aspirated from other tissue, showing 100% accuracy with no false positives or false negatives.

The combination of the cassette module 20 and reader 40 is designed to be an adjunct in surgeon decision making and not a diagnostic tool. Its use in the operating room is in the context of the surgeon's active visual identification of parathyroid glands and use of all other tools available. It is also notable that in thyroid surgery, identification of normal parathyroid glands is often critical to prevent their removal by accident. In these cases, serum parathyroid assays are not generally available, making the only alternative a biopsy and histopathologic diagnosis. The rapid lateral flow immunoassay test kit described herein is therefore also very helpful in performing a Thyroidectomy.

In-office diagnosis of parathyroid adenoma current practice includes performing ultrasound guided needle aspiration of a suspected gland and to send the specimen to the lab for diagnosis based on the quantitative PTH level.

The unique clinical circumstance for use of the combination of the cassette module 20 and reader 40 is that it augments the surgical task by providing the surgeon with additional information. All oversight and review of the task of visual identification, use of other assistive devices, review of serum PTH levels after gland removal, and subsequent pathologic examinations are unchanged by the addition of the combination of the cassette module 20 and reader 40 to the surgeon's toolkit.

Biopsy injures the gland and delays the procedure while the specimen is processed and review by the pathologist. Innovative surgeons have realized that the tests for intraoperative measurement of serum parathyroid hormone (IOPTH), if loaded with needle aspirations of the glands, can be configured to yield identification results approaching that of biopsy and histopathologic examination in reliability.

We claim:

1. A rapid parathormone identification and test kit for use in locating parathyroid glands and providing quantitative or semiquantitative measurement of parathormone in serum PTH comprises a test module comprising an immune-chromatographic lateral flow assay cassette configured to receive a needle aspirated sample from target tissue wherein said test module comprises:
a. anti-parathyroid antibody conjugated to nanoparticles deposited on a conjugate pad,
b. a pad membrane in the test module for receiving the liquid aspiration sample of tissue injected through a sample port so as to provide capillary flow along the membrane so that the sample flows into the conjugate pad and binds with the anti-parathyroid on the conjugate to form a sample/conjugate mixture,
c. a third membrane to receive the sample/conjugate mixture,
d. a test line comprising anti-parathyroid, said test line providing a readable indication of a positive presence of parathormone when a sufficient quantity thereof is present in the aspiration sample, and
e. a control line containing a host animal antibody.

2. The rapid parathormone identification and test kit of claim 1 wherein the readable indication on the test line is a positive signal that the sample was taken from a parathyroid gland.

3. The rapid parathormone identification and test kit of claim 2 wherein a readable indication on the control line indicates that the test was executed correctly even if a positive signal was not exhibited at the test line.

4. The rapid parathormone identification and test kit of claim 1 wherein the readable indication on the test line is readable either visually or by digital reader.

5. The rapid parathormone identification and test kit of claim 1 configured to provide an indication of a quantity of parathormone in a delivered sample of serum PTH within 5 minutes, said test kit providing an indication in said 5 minutes that less than about 30 pg/ml parathormone is in the aspirated sample, or parathormone is present in concentrations of from 30 pg/ml to at least about 250 pg/ml, indicating that the serum PTH sample was likely aspirated from a parathyroid gland.
